(19) United States
(12) Patent Application Publication
(10) Pub. No.: US 2017/0112886 A1
(43) Pub. Date: Apr. 27, 2017

(54) COMPOSITION CONTAINING ARONIA BERRY EXTRACT, RED SPINACH EXTRACT AND BEETROOT EXTRACT

(71) Applicant: Barbara Depta, Palm Beach Gardens, FL (US)

(72) Inventor: Barbara Depta, Palm Beach Gardens, FL (US)

(21) Appl. No.: 15/299,887

(22) Filed: Oct. 21, 2016

Related U.S. Application Data
(60) Provisional application No. 62/244,334, filed on Oct. 21, 2015.

(51) Int. Cl. A61K 36/21; A61K 36/73

(52) U.S. Cl. CPC A61K 36/21; A61K 36/73

(57) ABSTRACT

The present disclosure relates to compositions including a composition comprising red spinach extract, red beet extract, and aronia berry extract that increase athletic energy and endurance and enhance whole-body and/or after-workout muscle recovery through increase in blood flow. The compositions also relate to decreasing acute body inflammation after exercise to promote healing processes throughout the body, wherein the compositions are used according to a dietary plan to improve human health, increase athletic energy and endurance, and enhance whole-body and/or muscle recovery through an increase in blood flow.

### PRIORITY STATEMENT

The present application claims priority to U.S. Provisional Application No. 62/244,334, filed on Oct. 21, 2015 entitled A Dietary Supplement Containing Red Spinach Extract and Beetroot Extract.

### FIELD OF THE INVENTION

Some embodiments in the present disclosure relate to compositions and to using the compositions to increase blood flow for energy, endurance, and body/muscle recovery in humans and, in particular, to uses of the composition related to human sports performance. The present disclosure also relates to decreasing acute body inflammation after exercise to promote healing processes throughout the body.

### SUMMARY OF THE INVENTION

The present disclosure relates to the use of a composition containing aronia berry extract, red spinach extract and red beet root extract in a combination to increase blood flow for endurance and muscle recovery. In some embodiments, the ingredients aronia berry extract, red spinach extract and red beet extract are used together as a composition in liquid form, pill form or powder form. In some embodiments, additional components can be added to the composition to provide color, flavor, texture, odor, etc.

### BACKGROUND OF THE INVENTION

When humans strain their bodies and minds, they suffer chronic inflammation, exhaustion, and diabetes. Some of the main culprits of these ailments are daily psychological stress, excessive exercise, lack of exercise, poor nutrition, sugar, carbohydrates, excessive work, and lack of sleep.

Nitric Oxide is essential and crucial for optimal health. Nitric Oxide helps to relax the blood vessels, promote artery dilation, and improve circulation. It also supports healthy blood pressure and increases nutrient and oxygen delivery to every system, organ, and tissue in the body, Nitric Oxide also helps the body support cardiovascular and heart health. Nitric Oxide impacts the modulation of skeletal muscle function, which impacts the regulation of blood flow, muscle contractility, glucose & calcium homeostasis, and mitochondrial respiration (energy production). Nitric Oxide keeps the endothelium (lining of the arteries) healthy. A healthy endothelium in turn helps produce Nitric Oxide. A person's diet impacts Nitric Oxide levels as Nitric Oxide is also produced in the mouth and the digestive tract. As a person ages, Nitric Oxide production falls.

### DETAILED DESCRIPTION

Some embodiments of the present disclosure include a composition comprising ingredients that increase blood flow for energy, for improving an individual's endurance, and for enhancing body/muscle recovery in athletes. In some embodiments, these benefits are realized by targeting acute body inflammation that occurs after a physical workout. This targeting increases blood flow to the inflamed area and promotes the healing processes throughout the body. The present disclosure relates to a composition, which in some embodiments is a dietary supplement that may be useful for increasing blood flow for energy, endurance, and after-workout body/muscle recovery and, in certain embodiments, for uses related to human sports performance. Also described in some embodiments is the method for using the composition, such as for increasing blood flow for energy, endurance, and after-workout body/muscle recovery and, in certain embodiments, for uses related to human sports performance. Some embodiments include kits, comprising the composition to be consumed according to a dietary regimen for increasing blood flow for energy, endurance, and after-workout body/muscle recovery in humans.

In some embodiments the composition comprises at least the following active ingredients: aronia berry extract, red spinach extract and red beet root extract in varying concentrations.

Red spinach is the most powerful Nitric Oxide enhancer. Red spinach increases Nitric Oxide five times more than red beets do. Red spinach delays lactate accumulation (allowing for longer workouts), modulates skeletal muscle function, dilates blood vessels, increases the flow of blood and oxygen to tissues, impacts the regulation of blood flow, enhances muscle contractility, improves glucose and calcium homeostasis, and improves mitochondrial respiration (which improves energy production). Red spinach is also high in potassium.

Red beet extract reduces blood pressure through the vasodilation mechanism and also increases the oxygenation of muscles during exercise. Red beet extract improves athletic endurance through better oxygenation of muscles, modulates skeletal muscle function, regulates blood flow, and improves mitochondrial respiration (which improves energy production).

Aronia berry (chokeberry) is an antioxidant powerhouse, containing catechin and the highest anthocyanin concentration of all berries. It provides the health benefits of combining red wine and green tea. Compounds found in chokeberries are known for their biological activity, including antioxidant and radical scavenging properties. Chokeberries help the body protect against atherosclerosis, hypertension, heart attack, stroke, and neurodegenerative diseases (i.e., chokeberries slow the aging process). The aronia berry is packed with powerful compounds that help persons with chronic disease or daily pain and persons who have undergone surgery to heal faster.

Some embodiments of the present disclosure promote a healthy cardiovascular system, prolonged endurance, and enhanced performance.

Some embodiments of the present disclosure have some of the following characteristics:
All natural
0 fat
No added sugar—provided by beet
No added oxalate—provided by spinach
14% DV of potassium
100% water soluble
Neutral pH
0 g of carbs
NO GMO
Gluten free—Kosher—Halal

Some embodiments of the present disclosure can be used by athletes to increase endurance, decrease inflammation, and enhance recovery. Some embodiments of the present disclosure can be used as a healthier option for increasing energy and as a natural option for health improvement. Some embodiments of the present disclosure can be used for those with diabetic or cardiovascular issues to stabilize blood-sugar levels, enhance blood-flow through the cardiovascular system, reduce oxidative stress, and lower systolic and diastolic blood pressure. Some embodiments of the present disclosure can be consumed daily for its preventive health benefits or as needed before or after intense physical activity.

In some embodiments the composition is provided in the form of a powdered drink. In certain embodiments, the powdered drink may include the ingredients aronia berry extract, red spinach extract and red beet extract. In some embodiments, the powder may further comprise additional active ingredients such as glutamine, glucosamine, branched chain amino acids, digestive enzymes, protein, collagen and sugar in various amounts.

In some embodiments the composition is provided in the form of a powdered drink where the powder may comprise additional inactive ingredients such as flavors, thickening agents, gelling agents, sweeteners, colorants, and preservatives.

In certain embodiments, the combination of the active ingredients aronia berry extract, red spinach extract and red beet extract provides increased blood flow when administered to human subjects, particularly vasodilation and antioxidant benefits. For example, the composition may have a positive effect on pre-exercise, recovery after strenuous exercise, vascular health, and connective tissue recovery. Combinations that add the additional ingredients may also have the same positive effects.

Certain embodiments of red spinach extract have high concentrations of nitrates. In fact, in some instances red spinach extract is a source of one of the highest concentrations of nitrates in nature. Nitrates are involved in vasodilation events throughout the human body.

Some human clinical studies have shown reduction in blood pressure by the vasodilating mechanism of red beet extract, as well as its oxygenation of muscles during exercise, thus enhancing exercise tolerance. Thus, red beet extract improves and supports the cardiovascular, respiratory, and metabolic systems throughout the body.

Aronia berry extract is packed with antioxidants and other nutrients such as phenolic compounds. Antioxidants protect against many degenerative diseases as well as promote healing in exercise-strained human bodies. Several clinical studies done in humans have shown an increase in endurance in athletes supplemented with aronia berry extract.

L-Glutamine is the most common amino acid found in the human muscle. During exercise glutamine stores are depleted and must be recovered. Glutamine plays many roles, especially in protein metabolism, cell volumizing and is anti-catabolic. It also supports new muscle growth as seen in several human clinical studies.

Glucosamine's main purpose is to create long chains of glycosaminoglycans that are building blocks for joint cartilage. Many human clinical studies have shown the benefit of supplementing with glucosamine on joint health as well as cartilage repair.

Branched chain amino acids are essential amino acids in the human body. They aid in the energy production and boost muscle growth. Clinical studies show that leucine plays the most critical role in muscle growth signaling and is required to repair and build muscles.

Digestive enzymes are required for a healthy digestive tract. A healthy gut also mirrors a healthy immune system. With a healthy gut the human body is able to absorb all the nutrients required to repair, grow, and maintain healthy muscles, tendons, joints, and cardiovascular system.

Proteins are required to build muscle. Many clinical studies have investigated the use of protein in recovery after exercise. Those studies show that proteins are an essential component in building muscle.

Sugar is a carbohydrate used for quick energy conversion. Studies have shown that sugars are used first as energy substrates and allow the cells to recover after strenuous exercise.

Collagen constitutes the building blocks of connective tissue, tendons, joints, cartilage, as well as skin and without them many connective disorders would be observable. Clinical studies supplementing with collagen have shown many beneficial effects on joint and cartilage health, as well as bone strength and wound healing. Collagen is also an essential component of blood vessels, thus strengthening the cardiovascular system.

In some embodiments, the composition comprises at least 2 of the active ingredients listed above. In some embodiments, the composition comprises at least 2, 3, 4, 5, 6, 7, 8, 9, or all of the active ingredients.

In some embodiments, the composition comprises at least 2 active ingredients that are synergistic in their effects. In some embodiments, the composition comprises at least 2, 3, 4, 5, 6, 7, 8, 9, or 10 synergistic active ingredients. For example, in certain embodiments the active ingredients may be synergistic with respect to their positive effects on increased blood flow for energy, endurance, and body/muscle recovery.

In some embodiments, the effects of 2 or more of the active ingredients are additive. In some embodiments, the effects of at least 2, 3, 4, 5, 6, 7, 8, 9, or 10 of the active ingredients are additive. For example, the effects may be additive with respect to having a positive effect on increased blood flow for energy, endurance, and body/muscle recovery.

In some embodiments, the effects of the at least 2-10 active ingredients are additive and synergistic.

In some embodiments, at least one of the active ingredients potentiates the positive effect on increased blood flow of at least one other active ingredient. For example, at least one active ingredient may potentiate the positive effect of at least one other ingredient on increased blood flow for energy, endurance, and body/muscle recovery.

In some embodiments, a powder comprises red spinach extract, red beet extract, Aronia berry extract, L-glutamine, glucosamine, branched chain amino acids, digestive enzymes, protein, collagen, and sugar. In some embodiments, the protein is from whey or vegetarian source. In some embodiments, glucosamine comes in the form of glucosamine hydrochloride, glucosamine sulfate, or N-acetyl glucosamine. In some embodiments, the ratio of the branched chain amino acids (Leu:Ile:Val) is 2:1:1. In some embodiments the sugar comes from ribose, fructose, or sucrose. In some embodiments the digestive enzymes are comprised of proteases and/or lipases or are imbedded in the protein, In some embodiments the collagen is from an animal or vegetarian source.

In some embodiments, the ingredients aronia berry extract, red spinach extract and red beet extract are used together as a composition in liquid form, pill form or powder form in a total quantity of approximately 2.6 g. For example, in some embodiments, the ingredients include red spinach extract in an amount ranging from 0.0001 to 2.6 g, red beet extract in an amount ranging from 0.0001 to 2.6 g and aronia berry extract in an amount ranging from 0.0001 to 2.6 g. As one of ordinary skill in the art will understand, the proportion of each of the three ingredients used in these embodiments can vary to achieve optimal results.

In some embodiments, the composition comprises 0.0001 g to 10 g red spinach extract, 0.0001 g to 10 g red beet extract and 0.0001 g to 10 g aroma berry extract.

In some embodiments, the active ingredients are provided in the following amounts:

Red spinach extract (0.0001-10 g);

Red beet extract (0.0001-10 g);

Aronia berry extract (0.0001-5 g);

L-Glutamine (0.0001-10 g);

Glucosamine (0.0001-2 g);

Branched chain amino acids (0.0001-5 g);

Digestive enzymes (0.0001-1 g);

Protein (0.0001-50 g);

Collagen (0.0001-10 g); and

Sugar (0.0001-10 g).

In some embodiments, the composition comprises 0.0001 g to 10 g red spinach extract.

In some embodiments, the composition comprises 0.0001 g to 10 g red beet extract.

In some embodiments, the composition comprises 0.0001 g to 5 g Aronia berry extract.

In some embodiments, the composition comprises 0.0001 g to 10 g L-glutamine.

In some embodiments, the composition comprises 0.0001 g to 2 g glucosamine.

In some embodiments, the composition comprises 0.0001 g to 5 g branched chain amino acids.

In some embodiments, the composition comprises 0.0001 g to 1 g digestive enzymes.

In some embodiments, the composition comprises 0.0001 g to 50 g protein.

In some embodiments, the composition comprises 0.0001 g to 10 g collagen.

In some embodiments, the composition comprises 0.0001 g to 10 g of sugar.

In a preferred embodiment, the composition increases blood flow for energy, endurance, and body/muscle recovery. In some embodiments, the composition supports health in other ways.

In some embodiments, the composition is an oral supplement. In some embodiments, the composition is an edible solid or an edible semi-solid. In certain embodiments, the composition is a powder reconstituted into potable liquid (i.e. water, etc.).

In some embodiments, the composition is taken for 1 week to 12 months. In some embodiments, the composition is taken for 1, 2, 3, or 4 weeks. In some embodiments, the composition is taken for 2, 3, 4, 5, 6, 7, 8, 9, 10, 11, or 12 months. In some embodiments, the composition is taken before exercise. In some embodiments, the composition is taken after exercise.

In some embodiments, additional components can be added to the composition to provide color, flavor, texture, odor, etc. In some embodiments, the composition comprises additional components that act as preservatives or stabilize one or more of the active ingredients. In some embodiments, the composition comprises additional components that improve absorption of one or more of the active ingredients.

In some embodiments, general observable improvements after taking the composition may include improvements to vascular health, improvements to muscle recovery, improvements to chronic body inflammation, improvements to muscle pain, and/or improvements to overall health.

In some embodiments, a work-out improvement kit is provided which comprises compositions according to some embodiments disclosed herein and a dietary plan comprising using the compositions in the kit to increase blood flow for energy, endurance and after workout body/muscle recovery.

In some embodiments, the kit comprises a composition that is an edible solid or an edible semi-solid. In a preferred embodiment, the composition in the kit is a powder reconstituted in a potable liquid.

The present disclosure may be embodied in other specific forms without departing from its spirit or essential characteristics. The described embodiments are to be considered in all respects only as illustrative and not restrictive.

What is claimed is:

1. A composition comprising:
red spinach extract,
red beet extract, and
aronia berry extract.

2. The composition of claim 1, wherein the ingredients comprise:
red spinach extract in an amount ranging from 0.0001 to 2.6 g;
red beet extract in an amount ranging from 0.0001 to 2.6 g; and
aronia berry extract in an amount ranging from 0.0001 to 2.6 g.

3. The composition of claim 1, wherein the composition totals approximately 2.6 g.

4. A powder for dissolving in a liquid drink comprising:
red spinach extract,
red beet extract, and
aronia berry extract.

5. The powder of claim 4, wherein the ingredients comprise:
red spinach extract in an amount ranging from 0.0001 to 2.6 g;
red beet extract in an amount ranging from 0.0001 to 2.6 g; and
aronia berry extract in an amount ranging from 0.0001 to 2.6 g.

6. A pill comprising:
an ingestible outer shell containing red spinach extract, red beet extract, and aronia berry extract.

7. The pill of claim 6, wherein the ingredients comprise:
red spinach extract in an amount ranging from 0.0001 to 2.6 g;
red beet extract in an amount ranging from 0.0001 to 2.6 g; and
aronia berry extract in an amount ranging from 0.0001 to 2.6 g.

8. A composition for increasing blood flow for energy, endurance, and body/muscle recovery comprising aronia berry extract, red spinach and red beet extracts.

9. A method for increasing blood flow for energy, endurance, and after-workout body/muscle recovery, in a human subject, the method comprising: administering to the subject a composition comprising red spinach extract, red beet extract, and aronia berry extract.

10. The method of claim 9, the method more specifically comprising administering to the subject red spinach extract in an amount ranging from 0.0001 to 2.6 g; red beet extract in an amount ranging from 0.0001 to 2.6 g; and aronia berry extract in an amount ranging from 0.0001 to 2.6 g.
